(19) United States
(12) Patent Application Publication
(10) Pub. No.: US 2008/0248134 A1
(43) Pub. Date: Oct. 9, 2008

(54) Oral compositions, use and combinations of N-[2-(dimethylamino)ethyl]-2,6 dimethyl-1-oxo-1,2-dihydrobenzo[b]-1,6-naphthyridine-4-carboxamide and closely related analogues thereof

(75) Inventors: Bruce Charles Baguley, Remuera (NZ); Elaine Shirley Marshall, Drury (NZ); Catherine Jean Drummond, Waitakere (NZ)

Correspondence Address: OCCHIUTI ROHLICEK & TSAO, LLP, 10 FAWCETT STREET, CAMBRIDGE, MA, 02138, US

(73) Assignee: Auckland Uniservices Limited, Auckland (NZ)

(21) Appl. No.: 12/062,732

(22) Filed: Apr. 4, 2008

Related U.S. Application Data
(63) Continuation-in-part of application No. 10/514,523, filed on May 4, 2005.
(60) Provisional application No. 60/909,959, filed on Apr. 4, 2007.

(51) Int. Cl. A61K 33/24; A61K 31/4375; A61P 35/00; C07D 471/04

(52) U.S. Cl. 424/649; 546/81; 514/292

(57) ABSTRACT

This invention relates to compositions including a compound of Formula I

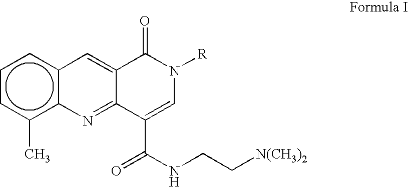

wherein R is selected from a C₁-C₆ alkyl, unsubstituted phenyl or phenyl substituted by one or more halo, C₁-C₆ alkyl or C₁-C₆ alkoxy, combinations of a compound of formula I with other chemotherapeutic agents, and the use of the compositions or combinations for the treatment of cellular proliferative disorders.

### CROSS-REFERENCE TO RELATED APPLICATIONS

This application is a continuation-in-part of U.S. Utility application Ser. No. 10/514,523, filed on May 4, 2005 and also claims the benefit of U.S. Provisional Application No. 60/909,959, filed on Apr. 4, 2007. The contents of both applications are hereby incorporated by reference in their entirety.

### BACKGROUND OF THE INVENTION

Despite progress in the chemotherapeutic treatment of cellular proliferative disorders including metastatic cancer, success in advanced cancers such as breast cancer, prostate cancer, colon cancer, small cell lung cancer and melanoma is still very limited. The class of cellular enzymes called topoisomerases constitutes an important target for selective chemotherapy because the enzymes are often elevated in tumour tissue. Topoisomerase-directed agents inhibit these enzymes, effectively subverting its action so that instead of maintaining the DNA they induce long-lived lesions that can lead to cell death. There are two main classes of topoisomerase inhibitors in current clinical use, type I topoisomerase inhibitors such as irinotecan and topotecan, and type II topoisomerase inhibitors such as doxorubicin and etoposide. Although both classes have widespread application in the treatment of metastatic cancer, a significant proportion of cancers are resistant to such treatment.

Targeting multidrug resistant cancers is one of the main aims of cancer drug development. The common mechanisms that produce multidrug resistance in cancer cells are decreased uptake of water-soluble drugs, cell changes that decrease the ability of cytotoxic drugs to kill cells and increased energy-dependent efflux of drugs from the cell (Szakacs, G et al. Targeting multidrug resistance in cancer. Nature Reviews Drug Discovery 2006, 5:219-234).

Most anticancer drugs enter cells by a passive diffusion process and only some (such as cisplatin, which utilises a copper transporter and nitrogen mustard, which utilises a choline transporter) enter by a facilitated process. In contrast, many anticancer drugs are susceptible to energy dependent efflux mechanisms that result in the concentration of free drug inside the cell being substantially lower than that outside the cell. It is therefore generally not possible to design drugs that are efficiently transported into cancer cells. There is a need to develop drugs that can evade these mechanisms.

Actinomycin D was the first successful DNA binding topoisomerase II inhibitor to be used clinically, but it did not have broad spectrum anticancer activity. Doxorubicin and related anthracyclines were the first to be widely used in clinical practice. Amsacrine, the first synthetic DNA binding drug targeting topoisomerase II to go into clinical practice, was developed in the Auckland Cancer Society Research Centre (ACSRC) (Cain B F, Atwell G J. The experimental antitumour properties of three congeners of the acridylmethanesulphonanilide (AMSA) series. European Journal of Cancer & Clincal Oncology 1974, 10:539-549) and is still in widespread use for the treatment of acute leukaemia (Arlin Z A. Current status of amsacrine (AMSA) combination chemotherapy programs in acute leukemia. Cancer Treatment Reports 1983, 67:967-970). Asulacrine was developed as a more potent and active analogue of amsacrine (Harvey V J et al. Phase II study of the amsacrine analogue CI-921 (NSC 343499) in non-small cell lung cancer. European Journal of Cancer 1991, 27:1617-1620). N-[2-(dimethylamino)ethyl]acridine-4-carboxamide dihydrochloride (DACA) was developed in a search for novel analogues (Atwell G J et al. Potential antitumor agents. 50. In vivo solid-tumor activity of derivatives of N-[2-(dimethylamino)ethyl]acridine-4-carboxamide. Journal of Medicinal Chemistry 1987, 30:664-669) and showed remarkable anticancer activity against the Lewis lung and Colon 38 murine tumours (Baguley B C et al. Experimental solid tumour activity of N-[2-(dimethylamino)ethyl]-acridine-4-carboxamide. Cancer Chemotherapy and Pharmacology 1995, 36:244-248). DACA was able to overcome resistance mediated by both P-glycoprotein and multidrug resistance protein (MRP) and overcame, at least partially, “atypical” multidrug resistance mediated by the type II topoisomerase enzyme (Finlay G J et al. In vitro assessment of N-[2-(dimethylamino)ethyl]acridine-4-carboxamide, a DNA-intercalating antitumour drug with reduced sensitivity to multidrug resistance. Cancer Chemotherapy and Pharmacology 1993, 31:401-406).

Further studies demonstrated that DACA acted simultaneously as a poison of the enzyme topoisomerase II and a catalytic inhibitor of topoisomerase I (Bridewell D J et al. Mechanism of cytotoxicity of N-[2-(dimethylamino)ethyl]acridine-4-carboxamide and of its 7-chloro derivative: the roles of topoisomerases I and II. Cancer Chemotherapy and Pharmacology 1999, 43:302-308). DACA thus appears to be a member of a new class of “dual” topoisomerase inhibitors (Denny W A, Baguley B C. Dual topoisomerase I/II inhibitors in cancer therapy. Current Topics in Medicinal Chemistry 2003, 3:1349-1364). DACA underwent phase I and phase II clinical trial but an unexpected dose-limiting side effect, where DACA induced an intense burning sensation, eventually led to the closing of the Phase II clinical trial (McCrystal et al. Phase I study of the cytotoxic agent N-[2-(dimethylamino)ethyl]acridine-4-carboxamide. Cancer Chemotherapy and Pharmacology 1999, 44:39-44).

Further development of these compounds lead to the production of XR 11576, a phenazine derivative that was approximately 4-fold more potent than DACA and showed evidence of dual topoisomerase I/II activity (Mistry P et al. In vitro and in vivo characterization of XR 11576, a novel, orally active, dual inhibitor of topoisomerase I and II. Anticancer Drugs 2002, 13:15-28).

More recently, a large number of new compounds differing chemically in many aspects from DACA were described in WO 03/097642, which disclosed the synthesis of a large series of benzonaphthyridinone derivatives. The disclosure of WO 03/097642 is incorporated herein in its entirety.

The ability of an anticancer drug to be administered orally is of great advantage to cancer chemotherapy in general. A disadvantage of the drug doxorubicin (and of related anthracyclines such as daunorubicin and epirubicin) is that they cannot be administered orally. Etoposide and teniposide, which also target the enzyme topoisomerase II, can be administered orally, however, they are susceptible to mulitdrug resistance and have a narrow clinical spectrum of activity. The antileukaemia drug amsacrine is not active orally and the related acridine derivative asulacrine was developed partly on the basis of its oral bioavailability. The investigational drug DACA was not active orally because of extensive liver metabolism.

It is an object of the present invention to provide novel compositions and combinations including N-[2-(dimethylamino)ethyl]-2,6 dimethyl-1-oxo-1,2-dihydrobenzo[b]-1,6-naphthyridine-4-carboxamide (Formula II) and closely related analogues thereof, and methods of use for the treatment and/or prevention of cellular proliferative disorders, or to at least provide a useful alternative.

### SUMMARY OF THE INVENTION

In a first aspect, the invention may provide a composition including a compound of Formula I

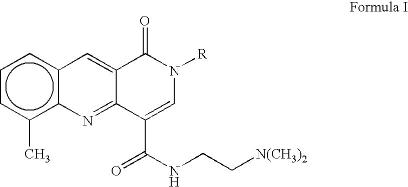

wherein R is selected from a C₁-C₆ alkyl, unsubstituted phenyl or phenyl substituted by halo, C₁-C₆ alkyl or C₁-C₆ alkoxy or an enantiomer, racemate, isomer or physiologically acceptable salt thereof.

The composition may be for use in the treatment of multidrug resistant cellular proliferative disorders and/or the treatment and/or prevention of abnormal or aberrant cell growth in a subject in need of such treatment.

Preferably, the composition includes a compound of Formula I wherein R is methyl, ethyl, butyl, unsubstituted phenyl, 4-fluoro-phenyl or 3,4-dimethoxy-phenyl. Most preferably, R is methyl (Formula II).

Preferably, the composition may be adapted to provide the compound of Formula I in an amount in the range of about 0.5 mg/kg of body weight of the subject to about 20 mg/kg of body weight of the subject.

More preferably, the amount may be in the range of about 0.5 mg/kg body weight of the subject to about 10 mg/kg body weight of the subject.

Preferably, the composition may further include a pharmaceutically acceptable excipient, adjuvant, carrier, buffer or stabiliser.

The pharmaceutically acceptable excipient, adjuvant, carrier, buffer or stabiliser should be non-toxic and should not interfere with the efficacy of the active ingredient. The precise nature of the carrier or other material will depend on the route of administration, which can be oral, or by injection, such as intraperitoneal, cutaneous, subcutaneous, or intravenous injection.

For intravenous, intraperitoneal, cutaneous or subcutaneous injection, the active ingredient will be in the form of a parenterally acceptable aqueous solution which is pyrogen-free and has a suitable pH, isotonicity and stability. Those of relevant skill in the art are well able to prepare suitable solutions. Preservatives, stabilisers, buffers antioxidants and/or other additives can be included as required.

A second aspect of the invention may provide an oral composition of a compound of Formula I as defined above.

The composition may be for use in the treatment of multidrug resistant cellular proliferative disorders and/or the treatment and/or prevention of abnormal or aberrant cell growth in a subject in need of such treatment.

Preferably, the oral composition includes a compound of Formula I wherein R is methyl, ethyl, butyl, unsubstituted phenyl, 4-fluoro-phenyl or 3,4-dimethoxy-phenyl. Most preferably, R is methyl.

Preferably, the oral composition may include the compound of Formula I in an amount in the range of about 0.5 mg/kg of body weight of the subject to about 20 mg/kg of body weight of the subject.

More preferably, the amount may be in the range of about 0.5 mg/kg body weight of the subject to about 10 mg/kg body weight of the subject.

Preferably, the composition may further include a pharmaceutically acceptable excipient, adjuvant, carrier, buffer or stabiliser.

The pharmaceutically acceptable excipient, adjuvant, carrier, buffer or stabiliser should be non-toxic and should not interfere with the efficacy of the active ingredient.

Preferably, the composition is formulated as a single dose unit.

Pharmaceutical compositions for oral administration may be in tablet, capsule, powder or liquid form. A tablet may include a solid carrier or an adjuvant. Liquid pharmaceutical compositions generally include a liquid carrier such as water, animal or vegetable oils, mineral oil or synthetic oil. Physiological saline solution, dextrose or other saccharide solution or glycols such as ethylene glycol, propylene glycol or polyethylene glycol can be included. A capsule may include a solid carrier such as gelatin.

A third aspect of the invention may provide a combination or composition of a compound of Formula I as defined above and one or more chemotherapeutic agents.

The combination may be for use in the treatment of multidrug resistant cellular proliferative disorders and/or the treatment and/or prevention of abnormal or aberrant cell growth in a subject in need of such treatment.

Preferably, the combination includes a compound of Formula I wherein R is methyl, ethyl, butyl, unsubstituted phenyl, 4-fluoro-phenyl or 3,4-dimethoxy-phenyl.

Preferably, the combination includes a compound of Formula I wherein R is methyl.

The list of preferred chemotherapeutic agents includes but is not limited to:

temozolomide or other DNA methylating agents;

cisplastin or other platinum-based derivatives;

cyclophosphamide or other DNA-alkylating agents;

doxorubicin, mitoxantrone, camptothecin or other topoisomerase inhibitors;

methotrexate, gemcitabine or other antimetabolites;

docetaxel or other taxanes; and

kinase inhibitors.

Preferably, the chemotherapeutic agent in the combination is temozolomide.

Most preferably, the combination includes a compound of Formula I wherein R is methyl (Formula II) and the chemotherapeutic agent is temozolomide.

Preferably, the combination includes a compound of Formula I in an amount in the range of about 0.5 mg/kg of body weight of the subject to about 20 mg/kg of body weight of the subject and temozolomide in an amount in the range of 100 to 300 mg daily.

More preferably, the combination includes a compound of Formula I in an amount range of about 0.5 mg/kg body weight of the subject to about 10 mg/kg body weight of the subject.

Preferably, the combination is orally available.

In a fourth aspect, the invention may provide a method of treating a subject with a multidrug resistant cellular proliferative disorder including the step of administering to a subject in need thereof a composition including a compound of Formula I as defined above.

Preferably, the cellular proliferative disorder may include for example, cancer(s) of the breast, lung, prostate, kidney, skin (including melanoma), neural, ovary, uterus, liver, pancreas, cervix, epithelial, gastric, intestinal, exocrine, endocrine, lymphatic, haematopoietic system or head and neck tissue.

More preferably, the cellular proliferative disorder may include leukaemia(s), lymphoma(s), multiple myeloma, sarcoma(s), and brain tumour(s), and cancer(s) of the lung, breast, ovary, testes, cervix and colon.

Preferably, the method defined above may further include the step of administering other chemotherapeutic agents, as defined above, before, during or after the administration of the composition of the present invention.

Alternatively, the method defined above may further include the step of administering radiotherapy to the tumour cells before, during or after the administration of the composition of the present invention.

In a fifth aspect, the invention may provide a method of treating a subject with a multidrug resistant cellular proliferative disorder including the step of administering to a subject in need thereof a combination including a compound of Formula I as defined above and another chemotherapeutic agent as defined above.

Preferably, the cellular proliferative disorder may include for example, cancer(s) of the breast, lung, prostate, kidney, skin (including melanoma), neural, ovary, uterus, liver, pancreas, cervix, epithelial, gastric, intestinal, exocrine, endocrine, lymphatic, haematopoietic system or head and neck tissue.

More preferably, the cellular proliferative disorder may include leukaemia(s), lymphoma(s), multiple myeloma, sarcoma(s), and brain tumour(s), and cancer(s) of the lung, breast, ovary, testes, cervix and colon.

In a sixth aspect the invention may provide a method of treating and/or preventing abnormal or aberrant cell growth in a subject including the step of administering to a subject in need thereof a composition including a compound of Formula I as defined above in an amount sufficient to treat and/or prevent the abnormal or aberrant cell growth.

Preferably, the abnormal or aberrant cell growth is a cellular proliferative disorder, which may include a neoplasm, a tumour or a cancer.

Preferably, the cellular proliferative disorder may include for example, cancer(s) of the breast, lung, prostate, kidney, skin (including melanoma), neural, ovary, uterus, liver, pancreas, cervix, epithelial, gastric, intestinal, exocrine, endocrine, lymphatic, haematopoietic system or head and neck tissue.

More preferably, the cellular proliferative disorder may include leukaemia(s), lymphoma(s), multiple myeloma, sarcoma(s), and brain tumour(s), and cancer(s) of the lung, breast, ovary, testes, cervix and colon.

Preferably, the amount sufficient may be in the range of about 0.5 mg/kg of body weight of the subject to about 20 mg/kg of body weight of the subject.

More preferably, the amount sufficient may be in the range of about 0.5 mg/kg body weight of the subject to about 10 mg/kg body weight of the subject.

Preferably, the method defined above may further include the step of administering other chemotherapeutic agents as defined above, before, during or after the administration of the composition according to the invention.

Alternatively, the method defined above may further include the step of administering radiotherapy to the tumour cells before, during or after the administration of the composition according to the invention.

In a seventh aspect, the invention may provide a method of treating and/or preventing abnormal or aberrant cell growth in a subject including the step of administering to a subject in need thereof a combination as defined above including a compound of Formula I as defined above and another chemotherapeutic agent.

Preferably, the abnormal or aberrant cell growth is a cellular proliferative disorder, which may include a neoplasm, a tumour or a cancer.

Preferably, the cellular proliferative disorder may include for example, cancer(s) of the breast, lung, prostate, kidney, skin (including melanoma), neural, ovary, uterus, liver, pancreas, cervix, epithelial, gastric, intestinal, exocrine, endocrine, lymphatic, haematopoietic system or head and neck tissue.

More preferably, the cellular proliferative disorder may include leukaemia(s), lymphoma(s), multiple myeloma, sarcoma(s), and brain tumour(s), and cancer(s) of the lung, breast, ovary, testes, cervix and colon.

In an eighth aspect of the invention, there may be provided the use of a composition including a therapeutically effective amount of a compound of Formula I as defined above for the manufacture of a medicament for treating and/or preventing a multidrug resistant cellular proliferative disorder.

Preferably, the cellular proliferative disorder may include for example, cancer(s) of the breast, lung, prostate, kidney, skin (including melanoma), neural, ovary, uterus, liver, pancreas, cervix, epithelial, gastric, intestinal, exocrine, endocrine, lymphatic, haematopoietic system or head and neck tissue.

More preferably, the cellular proliferative disorder may include leukaemia(s), lymphoma(s), multiple myeloma, sarcoma(s), and brain tumour(s), and cancer(s) of the lung, breast, ovary, testes, cervix and colon.

Preferably, the medicament is adapted for oral administration.

Preferably, the therapeutically effective amount of active ingredient in the medicament may be in the range of about 0.5 mg/kg of body weight of the subject to about 20 mg/kg of body weight of the subject.

More preferably, the therapeutically effective amount of active ingredient in the medicament may be in the range of about 0.5 mg/kg body weight of the subject to about 10 mg/kg body weight of the subject.

Preferably, the medicament may be adapted for administration in conjunction with other chemotherapeutic agents as defined above, before, during or after the administration of the medicament.

Alternatively, the medicament may be adapted for administration in conjunction with radiotherapy to the tumour cells before, during or after the administration of the medicament.

In a ninth aspect of the invention, there may be provided the use of a combination as defined above for the manufacture of a medicament for treating and/or preventing a multidrug resistant cellular proliferative disorder.

Preferably, the cellular proliferative disorder may include for example, cancer(s) of the breast, lung, prostate, kidney, skin (including melanoma), neural, ovary, uterus, liver, pancreas, cervix, epithelial, gastric, intestinal, exocrine, endocrine, lymphatic, haematopoietic system or head and neck tissue.

More preferably, the cellular proliferative disorder may include leukaemia(s), lymphoma(s), multiple myeloma, sarcoma(s), and brain tumour(s), and cancer(s) of the lung, breast, ovary, testes, cervix and colon.

In a tenth aspect of the invention, there may be provided the use of a composition including a therapeutically effective amount of a compound of Formula I as defined above for the manufacture of a medicament for treating and/or preventing abnormal or aberrant cell growth.

Preferably, the abnormal or aberrant cell growth is a cellular proliferative disorder, which may include a neoplasm, a tumour or a cancer.

Preferably, the cellular proliferative disorder may include for example, cancer(s) of the breast, lung, prostate, kidney, skin (including melanoma), neural, ovary, uterus, liver, pancreas, cervix, epithelial, gastric, intestinal, exocrine, endocrine, lymphatic, haematopoietic system or head and neck tissue.

More preferably, the cellular proliferative disorder may include leukaemia(s), lymphoma(s), multiple myeloma, sarcoma(s), and brain tumour(s), and cancer(s) of the lung, breast, ovary, testes, cervix and colon.

Preferably, the medicament is adapted for oral administration.

Preferably, the therapeutically effective amount of active ingredient in the medicament may be in the range of about 0.5 mg/kg of body weight of the subject to about 20 mg/kg of body weight of the subject.

More preferably, the therapeutically effective amount of active ingredient in the medicament may be in the range of about 0.5 mg/kg body weight of the subject to about 10 mg/kg body weight of the subject.

Preferably, the medicament may be adapted for administration in conjunction with other chemotherapeutic agents as defined above, before, during or after the administration of the medicament.

Alternatively, the medicament may be adapted for administration in conjunction with radiotherapy to the tumour cells before, during or after the administration of the medicament.

In an eleventh aspect of the invention, there may be provided the use of a combination as defined above for the manufacture of a medicament for treating and/or preventing abnormal or aberrant cell growth.

Preferably, the abnormal or aberrant cell growth is a cellular proliferative disorder, which may include a neoplasm, a tumour or a cancer.

Preferably, the cellular proliferative disorder may include for example, cancer(s) of the breast, lung, prostate, kidney, skin (including melanoma), neural, ovary, uterus, liver, pancreas, cervix, epithelial, gastric, intestinal, exocrine, endocrine, lymphatic, haematopoietic system or head and neck tissue.

More preferably, the cellular proliferative disorder may include leukaemia(s), lymphoma(s), multiple myeloma, sarcoma(s), and brain tumour(s), and cancer(s) of the lung, breast, ovary, testes, cervix and colon.

A twelfth aspect of the present invention may provide a method of inducing p53 expression and decreasing survivin expression in cells demonstrating abnormal or aberrant growth, by exposing the cells to a composition or combination according to the present invention.

Preferably, the abnormal or aberrant growth demonstrated by the cells is a cellular proliferative disorder, which may include a neoplasm, a tumour or a cancer.

Preferably, the cellular proliferative disorder may include for example, cancer(s) of the breast, lung, prostate, kidney, skin, neural, ovary, uterus, liver, pancreas, cervix, epithelial, gastric, intestinal, exocrine, endocrine, lymphatic, haematopoietic system or head and neck tissue.

More preferably, the cellular proliferative disorder may include leukaemia(s), lymphoma(s), multiple myeloma, sarcoma(s), and brain tumour(s), and cancer(s) of the lung, breast, ovary, testes, cervix and colon.

Preferably, in one embodiment of the present invention, the cells demonstrating abnormal or aberrant growth are located in vitro.

Alternatively, in another embodiment, the exposure may include the further step of administering to a subject in need thereof a composition or combination according to the present invention in an amount sufficient to induce p53 expression and decrease survivin expression in a subject having a cellular proliferative disorder.

Preferably, the amount sufficient may include a compound of Formula I in the range of about 0.5 mg/kg of body weight of the subject to about 20 mg/kg of body weight of the subject.

More preferably, the amount sufficient may be in the range of about 0.5 mg/kg body weight of the subject to about 10 mg/kg body weight of the subject.

A thirteenth aspect of the present invention provides for the use of a composition or combination of the present invention for the production of a medicament to increase p53 expression and decrease survivin production in a subject with a cellular proliferative disorder.

Preferably, the cellular proliferative disorder may include for example, cancer(s) of the breast, lung, prostate, kidney, skin (including melanoma), neural, ovary, uterus, liver, pancreas, cervix, epithelial, gastric, intestinal, exocrine, endocrine, lymphatic, haematopoietic system or head and neck tissue.

More preferably, the cellular proliferative disorder may include leukaemia(s), lymphoma(s), multiple myeloma, sarcoma(s), and brain tumour(s), and cancer(s) of the lung, breast, ovary, testes, cervix and colon.

A fourteenth aspect of the present invention provides a composition that provides up to 450 mg/m² of a compound of Formula I:

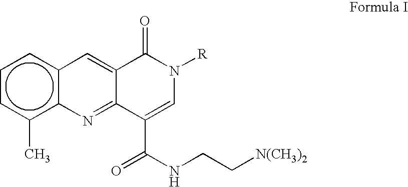

wherein R is selected from a C₁-C₆ alkyl, unsubstituted phenyl or phenyl substituted by halo, C₁-C₆ alkyl or C₁-C₆ alkoxy or an enantiomer, racemate, isomer or physiologically acceptable salt thereof.
Preferably, the composition further includes one or more chemotherapeutic agents.
Preferably, in the compound of Formula I, R is methyl and the composition further includes the chemotherapeutic agent temozolomide.
In a fifteenth aspect of the invention, there is provided a composition including a compound of Formula I:

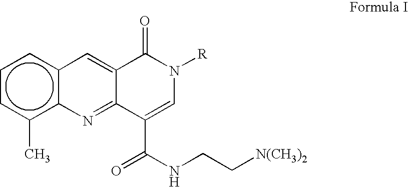

wherein R is selected from a C₁-C₆ alkyl, unsubstituted phenyl or phenyl substituted by halo, C₁-C₆ alkyl or C₁-C₆ alkoxy or an enantiomer, racemate, isomer or physiologically acceptable salt thereof, wherein the composition provides at least a 3-fold concentration differential in tumour tissue to non-tumour tissue or plasma concentration of compound of Formula I.
Preferably, the composition further includes one or more chemotherapeutic agents.
Preferably, in the compound of Formula I, R is methyl and the composition further includes the chemotherapeutic agent temozolomide.
In a sixteenth aspect of the invention, there is provided a method of treating and/or preventing abnormal or aberrant cell growth in a subject including the step of administering to a subject in need thereof a composition that provides up to 450 mg/m² of a compound of Formula I:

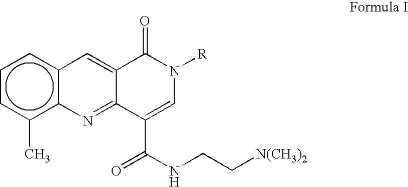

wherein R is selected from a C₁-C₆ alkyl, unsubstituted phenyl or phenyl substituted by halo, C₁-C₆ alkyl or C₁-C₆ alkoxy or an enantiomer, racemate, isomer or physiologically acceptable salt thereof.
In a seventeenth aspect, there is provided a method of treating a subject with a multidrug resistant cellular proliferative disorder, including the step of administering to a subject in need thereof a composition as defined in the fourteenth aspect, which further includes one or more chemotherapeutic agents.
In an eighteenth aspect, there is provided a method of treating and/or preventing abnormal or aberrant cell growth in a subject including the step of administering to a subject in need thereof a composition of Formula I as defined in the fourteenth aspect; wherein the composition provides at least a 3-fold concentration differential in tumour tissue to non tumour tissue or plasma concentration of compound of Formula I.
In a nineteenth aspect, there is provided a method of inducing p53 expression and decreasing survivin expression in cells demonstrating abnormal or aberrant growth, by exposing the cells to a compound of Formula I, whereby the exposure comprises administering to a subject in need thereof a composition including a compound of Formula I as defined in the fourteenth aspect, in an amount sufficient to induce p53 expression and decrease survivin expression in the subject having a cellular proliferative disorder.

The term “subject” as used herein refers to any animal having a disease or condition which requires treatment with a pharmaceutically-active agent. The subject may be a mammal, preferably a human, or may be a non-human primate or non-primates such as used in animal model testing. While it is particularly contemplated that the compounds of the invention are suitable for use in medical treatment of humans, it is also applicable to veterinary treatment, including treatment of companion animals such as dogs and cats, and domestic animals such as horses, ponies, donkeys, mules, llama, alpaca, pigs, cattle and sheep.

The term “C₁-C₆ alkyl” refers to straight chain, branched or cyclic hydrocarbon groups having from 1 to 6 carbon atoms. The term “C₁-C₆ alkoxy refers to straight chain or branched oxy-containing radicals each having alkyl portions of 1 to about 6 carbon atoms.

The term “halo” refers to fluorine, chlorine, bromide or iodine.

As used herein, the term “therapeutically effective amount” is meant an amount effective to yield a desired therapeutic response, for example, to prevent or treat a cellular proliferative disorder.

“Pharmaceutically acceptable carrier” means a pharmaceutically acceptable solvent, suspending agent or vehicle for delivering the compounds of the present invention.

“Cellular proliferative disorder” means that a cell or cells demonstrate abnormal growth, typically aberrant growth, leading to a neoplasm, tumour or a cancer. These disorders may include, for example, cancer(s) of the breast, lung, prostate, kidney, skin (including melanoma), neural, ovary, uterus, liver, pancreas, cervix, epithelial, gastric, intestinal, exocrine, endocrine, lymphatic, haematopoietic system or head and neck tissue.

Generally, neoplastic diseases are conditions in which abnormal proliferation of cells results in a mass of tissue called a neoplasm or tumour. Neoplasms have varying degrees of abnormalities in structure and behaviour. Some neoplasms are benign while others are malignant or cancerous. An effective treatment of neoplastic disease would be considered a valuable contribution to the search for cancer preventative or curative procedures. The compounds of the present invention may preferably be used in the treatment of leukaemia(s), lymphoma(s), multiple myeloma, sarcoma(s), and brain tumour(s), and for cancer(s) of the lung, breast, ovary, testes, cervix and colon.

“Multidrug resistance” refers to the ability of cancer cells to resist the effects of chemotherapeutic agents by means of energy-dependent efflux mechanism that reduces the intracellular concentration of the chemotherapeutic agent.

It is to be understood that the composition, combination or medicament of the present invention may be administered alone or in conjunction with other chemotherapeutic agents or treatments, especially radiotherapy, either simultaneously or sequentially, depending upon the condition to be treated.

It is to be understood that further aspects of the invention may become apparent from the following description with reference to the Figures and Examples given by way of example only.

### BRIEF DESCRIPTION OF THE FIGURES

FIG. 1 shows induction of p53 protein by a compound of Formula II, and comparisons to doxorubicin and etoposide.

FIG. 2 shows the inhibition of survivin production by a compound of Formula II in HCT 116 human colon cells and comparisons to doxorubicin and etoposide.

FIG. 3 shows the effect of a single oral dose of a compound of Formula II on colon 38 tumours.

FIG. 4 shows the response of NZM3 xenografts to treatment with a compound of Formula II, temozolomide or a combination of both agents.

FIG. 5 shows the response of a xenograft of a melanoma established directly from a patient with metastatic melanoma to treatment with a compound of Formula II.

FIG. 6 shows plasma pharmacokinetics of a compound of Formula II.

FIG. 7: Plasma concentration and Liver concentration profiles of a compound of Formula II

FIG. 8: Concentration profile of compound of Formula II in various tissues and plasma over time.

FIG. 9: Concentration profile of compound of Formula II in various tissues and plasma over time.

FIG. 10: Concentration of compounds, including a compound of Formula II, in tumour tissue versus plasma and liver tissue as a function of time.

FIG. 11: Ratio of tumour tissue/liver tissue AUC for various cancer treating compounds, including a compound of Formula II

FIG. 12: Predicted expected human dose of a compound of Formula II for use in clinical trials, based on the reported pharmacokinetic data and data from related therapies.

FIG. 13: Response of colon 38 tumours to the compound of Formula II in Rag1 immunodeficient mice and immunocompetent mice.

### DETAILED DESCRIPTION OF THE INVENTION

A composition, combination or medicament according to the present invention may be prepared by methods well known in the art of pharmacy. The compounds of the present invention may be presented in unit dosage form. Each carrier, diluent, adjuvant and/or excipient must be pharmaceutically “acceptable” in the sense of being compatible with the other ingredients of the composition and not injurious to the subject. Compositions include those suitable for oral, rectal, nasal, topical (including buccal and sublingual), vaginal or parenteral (including subcutaneous, intramuscular, intravenous and intradermal) administration. The compositions may conveniently be presented in unit dosage form and may be prepared by methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

The compounds of the present invention may also be used for the treatment and/or prevention of cellular proliferative disorders as described above. Generally, the terms “treating”, “treatment” and the like are used herein to mean affecting a subject, tissue or cell to obtain a desired pharmacological and/or physiological effect. The effect may be prophylactic in terms of completely or partially preventing a disease or symptoms thereof, and/or may be therapeutic in terms of a partial or complete cure of a disease. “Treating” as used herein covers any treatment of, or prevention of disease in a vertebrate, a mammal, particularly human, and includes: (a) preventing the disease from occurring in a subject that may be predisposed to the disease, but has not yet been diagnosed as having it; (b) inhibiting the disease, i.e., arresting its development; or (c) relieving or ameliorating effects of the disease, i.e., causing regression of the effects of the disease.

The pharmaceutical compositions according to the invention may be administered locally or systemically in a therapeutically effective dose. Amounts effective for this use will, of course, depend on the severity of the disease and the weight and general state of the subject.

Typically, dosages used in vitro may provide guidance in the amounts useful for in situ administration of the pharmaceutical composition, and animal models may be used to determine effective dosages for treatment of the cytotoxic side effects. Various considerations are described, e.g., in Langer, Science, 249: 1527, (1990).

The compounds of the present invention may be formulated as an oral formulation. Formulations for oral use may be in the form of hard gelatin capsules wherein the active ingredient is mixed with an inert solid diluent. They may also be in the form of soft gelatine capsules wherein the active ingredient is mixed with water or an oil medium or may be in the form of a liquid. The oral formulations may be prepared and administered as solid dose units, which may include tablets and capsules. The formulation may contain a therapeutically effective amount of the compound of the invention and optionally pharmaceutically acceptable carriers. Amounts effective for this use, depend on the severity of the disease and the weight and general state of subject. The dosages used may be in the range of about 0.5 mg/kg body weight of the subject to about 20 mg/kg body weight of the subject. The preferred dose range may be in the range of about 0.5 mg/kg body weight of the subject to about 10 mg/kg body weight of the subject.

The oral composition according to the present invention may be in the form of tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable preparations.

Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable preservatives include sodium benzoate, vitamin E, alphatocopherol, ascorbic acid, methyl paraben, propyl paraben or sodium bisulphite.

Suitable lubricants include magnesium stearate, stearic acid, sodium oleate, sodium chloride or talc. Suitable time delay agents include glyceryl monostearate or glyceryl distearate. The tablets contain the active ingredient in admixture with non-toxic pharmaceutically acceptable excipients which are suitable for the manufacture of tablets.

These excipients may be, for example, (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed. Coating may also be performed using techniques described in the U.S. Pat. Nos. 4,256,108; 4,160,452; and 4,265,874 to form osmotic therapeutic tablets for control release.

It will be understood, however, that the specific dose level and type of formulation for any particular subject will depend upon a variety of factors including the age, body weight, general health, sex, diet, time of administration, rate of excretion and the severity of the particular cellular proliferative disease undergoing therapy.

It has been surprisingly found that the combination of a compound of the present invention with the chemotherapeutic agent temozolomide demonstrates increased efficacy without increasing toxicity to the subject.

Unlike most anticancer drugs discussed previously, DNA binding anticancer drugs such as doxorubicin, amsacrine, DACA and compounds of Formula II may accumulate in both normal and cancer tissue as a result of DNA binding. At equilibrium, the free drug concentration inside the cell will not be higher than that outside the cell, but the total drug concentration may be substantially higher. Thus, the ratio of concentrations of cell-associated drug to external drug is typically 20 for amsacrine (Robbie M A, Baguley B C, Denny W A, Gavin J B, Wilson W R. Mechanism of resistance of non-cycling mammalian cells to 4′-(9-acridinylamino)-methane-sulphon-m-anisidide: comparison of uptake, metabolism and DNA breakage in log- and plateau-phase Chinese hamster fibroblast cell cultures. Cancer Res 1988; 48: 310-319) and 550 for DACA (Haldane A, Finlay G J, Hay M P, Denny W A, Baguley B C. Cellular uptake of N-[2-(dimethylamino)-ethyl]-acridine-4-carboxamide (DACA). Anti-Cancer Drug Design 1999; 14: 275-280).
An aim of selective cancer therapy is to produce a higher concentration of cell-associated drug in cells of tumour tissue than in cells of normal tissue. Uptake of a drug into tumour tissue is a function of firstly drug diffusion from the blood supply to the cell and secondly of diffusion through the plasma membrane into the cell. Because of the comparatively low vascular density of tumour relative to normal tissue (i.e. a comparatively long average diffusion distance from the blood vessel to the tumour cell), drug uptake by tumour tissue is almost invariably slower than that into normal tissue.
An advantage of a high DNA binding constant is that once in the cell, the drug is retained for a longer time. However, for most drugs, this theoretical advantage is offset by a correspondingly low rate of cellular uptake. For example, the clinical drug doxorubicin has a high DNA binding constant but a low rate of uptake into cells.
It has been surprisingly found that a compound of the present invention has the property of being taken up relatively efficiently by tumour tissue but, more importantly, to be retained selectively by tumour tissue in comparison to normal tissues. The half life or concentration differential of a compound of the present invention is significantly higher in tumour tissue than non tumour tissue. Thus the tissue half-life in mice is approximately 3 hours in plasma, liver, kidney, brain and heart tissue, but is approximately 9 hours in colon 38 tumour tissue. This is approximately a three-fold increase in the concentration differential in tumour tissue to non-tumour tissue and plasma concentration of Formula II.
The known DNA binding properties of a compound of Formula II, together with the pharmacokinetic observations suggesting that most of the administered drug is present on the DNA of normal and tumour tissue can be used to predict the expected human dose in clinical trials.
As previously stated, the ability of an anticancer drug to be administered orally is of great advantage to cancer chemotherapy in general. An unexpected feature of a compound of Formula II is that it has good bioavailability. For example, the time-concentration profiles for the different routes of administration (such as intraperitoneal, intravenous and oral) are quite comparable. As expected from the rate of gastric emptying, the initial increase in plasma and liver drug concentrations occur later after oral administration than they do after intravenous or intraperitoneal administration, but the plasma and tissue half-lives are similar.
These multiple factors result in a composition including a compound of formula I having surprising potency and efficacy with an expected intravenous dose of up to 450 mg (265 mg/m² surface area or 6.4 mg/kg body weight) against abnormal or aberrant cell growth and cellular proliferative disorders including metastatic cancer. Note: the mg/m² dosage units relate to the dose of drug in mg per square meter of skin surface area, which is a commonly used basis for dosing of cytotoxic drugs. A typical subject has a body weight of 70 kg and a surface area of 1.7 m². However, it will be appreciated that smaller subjects and larger subjects may have skin surface areas well outside this average. As such, the total dose required to treat a patient may vary significantly. In particular, the compounds of Formula I have surprising potency and efficacy against mulitdrug resistant cellular proliferative disorder.

### EXAMPLES

### Example 1

### In Vitro Cell Proliferation Assays

The concentrations of the compound of Formula II needed to inhibit cell growth by 50% (IC₅₀ values, nanomolar concentrations) were determined for different cells lines in continuous drug exposure assays (3-5 days depending on the growth rate of the cell lines). The compound was compared to several other drugs and was found to be highly potent (Table 1). The results with HCT116 cell lines show that the presence of a p53 pathway confers a slight increase in sensitivity. The results with the JL_{A} and JL_{D} lines, which have reduced topoisomerase II content as compared to the parental Jurkat line, indicate that the IC₅₀ value of the compound is increased with decreased topoisomerase content. This is also the case for etoposide, amsacrine, DACA and doxorubicin. This result suggests that topoisomerase II is at least one of the targets of the compound of the present invention.

TABLE 1
IC₅₀ values of a compound of Formula II and comparative compounds

| | Formula | | | | |
|---|---|---|---|---|---|
| Cell line | II | Etoposide | Amsacrine | DACA | Doxorubicin |
| HCT116p53⁺ | 8.6 | 210 | 25 | 360 | 10.5 |
| HCT116p53⁻ | 11 | 390 | 52 | 510 | 21 |
| Jurkat | 6.7 | 160 | 37 | 580 | 9.6 |
| JL_{A} | 38 | 2080 | 3100 | 1100 | 42 |
| JL_{D} | 53 | 14400 | 2700 | 2500 | 270 |

### Example 2

### In Vitro Susceptibility to Multidrug Resistance

Multidrug resistance mediated by p-glycoprotein and related transport proteins is an important means by which cancer cells evade cytotoxic drugs. The susceptibility of the compound of Formula II, to multidrug resistance was determined by growing two Chinese Hamster cell lines, one exhibiting no multidrug resistance (AuxB1) and one exhibiting significant resistance (CHrC5). IC₅₀ values of several drugs were measured in continuous exposure assays in these two cell lines. The resistance factor was calculated as the ratio of these values. High values indicate high susceptibility to resistance. The results (Table 2) show that the compound according to the invention has a low susceptibility to drug resistance.

TABLE 2
Resistance factors for Formula II and comparative compounds

| Cell line | Formula II | Etoposide | Amsacrine | DACA | Doxorubicin |
|---|---|---|---|---|---|
| AuxB1 | 3.5 | 1190 | 19 | 125 | 53 |
| CHrC5 | 5.2 | 8600 | 110 | 216 | 6100 |
| Resistance | 1.5 | 7.2 | 5.6 | 1.6 | 114 |
| factor | | | | | |

### Example 3

### In Vitro Cell Survival Assays

IC₅₀ values do not directly measure the cytotoxic potential of drugs because growth inhibition assays reflect cell cycle slowing as well as induced cell death. Two HCT116 human colon carcinoma lines, one with TP53 wild type status and one with TP53 mutant status, were exposed to the compound of Formula II for 6 hours. The cells were washed free of drug and surviving cells were measured by clonogenic assay. The concentration for maximal reduction of cell survival was 1% for p53 wild-type cells and 5% for p53 mutant cells. The cytotoxicity of the compound of the present invention was also tested in three Jurkat leukaemia lines, two of which, JL_{A} and JL_{D}, are resistant to topoisomerase inhibitors because of low cellular levels of topoisomerase II. The survival at a drug concentration of 0.4 μM (one hour exposure) was 28% for the wild-type Jurkat line and 100% for the resistant lines, consistent with the IC₅₀ results and supporting a role for topoisomerase II in the action of the compounds of Formula II.

### Example 4

### Induction of Cell Cycle Arrest

Flow cytometry was used to determine the magnitude and duration of cell cycle arrest induced by the compound of the present invention. Two human melanoma lines were used in a preliminary study, one having wild-type p53 status (NZM6) and one having mutant p53 status (NZM4). Cells were exposed to the compound of Formula II for one hour at a concentration of 0.3 μM, washed free of drug and grown for up to a further 9 days. The drug induced extended arrest of NZM6 cells in both G₁- and G₂-phase for the whole period of observation, while in NZM4 cells it showed minimal effect in G₁-phase but an extended effect in G₂-phase. These results show that the compounds of the present invention are capable of inducing long-term cell cycle arrest in at least some human melanoma lines.

### Example 5

### Induction of the p53 Pathway in Cultured Human Cancer Cells

The p53 protein is thought to play a major role in the induction of apoptosis in tumour cells (Levine A J. p53, the cellular gatekeeper for growth and division. Cell. 1997. 88(3):323-331). The p53 protein is a transcriptional activator, which acts in response to DNA damage and other stress pathways, and increases expression of proteins such as Fas and p21^{WAF1}. Cultures of the HCT116 human colon carcinoma line with TP53 wild type status were exposed to the compound of Formula II for 24 hours, protein extracts were prepared and the proteins in the extracts were separated by gel electrophoresis. Products were detected by western blotting. The compound of Formula II, as well as etoposide and doxorubicin, induced p53, p21 and Fas in dose-dependent fashions but the compound of the present invention provided a much greater response (FIG. 1). Moreover, at earlier times (4-10 hours) the compound of Formula II induced these proteins while etoposide and doxorubicin did not. A clear differential was also seen when the cell line was grown for 24 hours at a higher cell density (approaching confluence on culture plates). Under these conditions, the compound of Formula II induced these proteins while etoposide and doxorubicin did not.

### Example 6

### Inhibition of Survivin Production in Cultured Human Cancer Cells

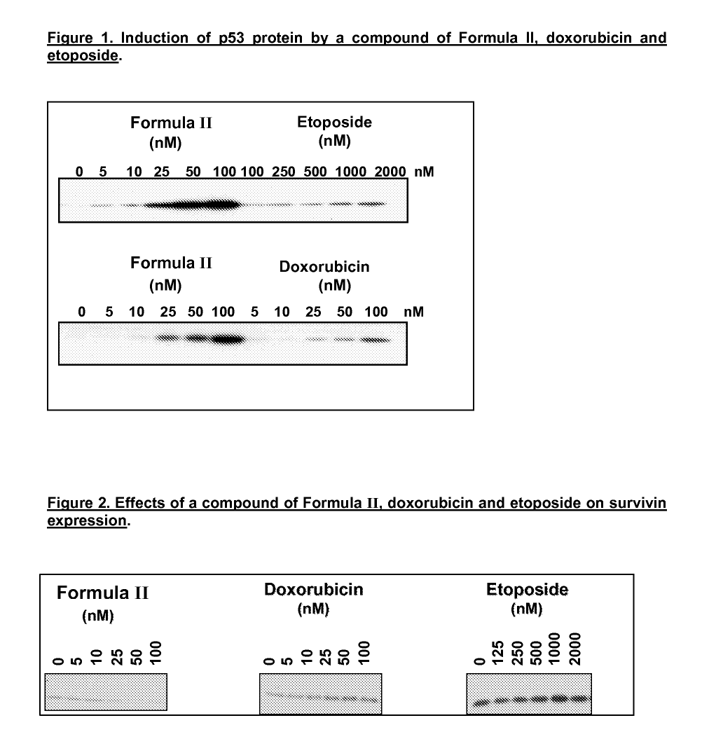

Survivin is a protein that is expressed selectively in many types of cancer cells and which is known to be associated with resistance to apoptosis (Zaffaroni N and Daidone M G. Survivin expression and resistance to anticancer treatments: perspectives for new therapeutic interventions. Drug Resistance Updates: Reviews and Commentaries in Antimicrobial and Anticancer Chemotherapy. 2002 5(2):65-72). The HCT116 human colon carcinoma line with TP53 wild type status was grown in culture under standard conditions and exposed to the compound of Formula II, doxorubicin or etoposide at various concentrations for 24 hours. Extracts were prepared, proteins were separated by electrophoresis and survivin expression was determined by western blotting. Exposure to the compound of Formula II was found to suppress survivin production while doxorubicin and etoposide had only small effects (FIG. 2).

### Example 7

### Induction of Tumour Growth Delays in Murine Tumours

The murine colon-38 carcinoma is a transplantable tumour that has been used extensively in drug development programmes to determine the activity of many different drugs (Goldin A et al. Current results of the screening program at the Division of Cancer Treatment, National Cancer Institute. European Journal of Cancer. 1981. 17(2):129-142). The tumour is implanted subcutaneously as 1-mm³ fragments and allowed to grow (8 days) to a tumour of approximately 3 mm in diameter before administration of test drugs.

The compound of Formula II was found to be orally active, with a single oral dose (8.9 mg/kg) inducing complete regression of colon 38 tumours (FIG. 3).

In addition, the compound of Formula II, by way of an intraperitoneal dose of 8.9 mg/kg, induced a growth delay of 8.5 days in a murine tumour (Lewis lung) that is also used extensively in drug development programmes.

### Example 8

### Tumour Growth Delays in Xenografts of Human Tumour Cell Lines

The compound of Formula II was evaluated in a number of xenografts using cell lines derived from cervical, endometrial, lung and colon carcinomas, leukaemia and melanoma (Table 3). These studies indicate that the compound according to the present invention has significant activity in xenografts.

TABLE 3
Activity of a compound of Formula II in human tumour xenografts

| | | | % | | |
|---|---|---|---|---|---|
| Tumour | Type | P53 | control | Activity* | Comparison |
| NZC1 | Cervix | Not | 38% | Active | Etoposide |
| | | known | | | active |
| NZEN1 | Endometrium | Not | 50% | Inactive | Doxorubicin |
| | | known | | | inactive |
| H460 | Lung | W.T. | 38% | Active | — |
| HCT116 | Colon | W.T. | 70% | Inactive | — |
| Jurkat LA | Leukaemia | Null | 28% | Active | Doxorubicin |
| | | | | | inactive |
| NZM3 | Melanoma | W.T. | 38% | Active | Etoposide |
| | | | | | active |
| NZM4 | Melanoma | Mutant | 90% | Inactive | Etoposide |
| | | | | | active |
| NZM10 | Melanoma | W.T. | 30% | Active | Etoposide |
| | | | | | active |
| NZMp1 | Melanoma | Not | <10% | Active | — |
| | | known | | | |
| NZMp2 | Melanoma | | 22% | Active | Etoposide |
| | | | | | active |

*NCI Criterion for activity tumour volume T/C ≦ 40%

### Example 9

### Response of a Xenograft to Combination Chemotherapy

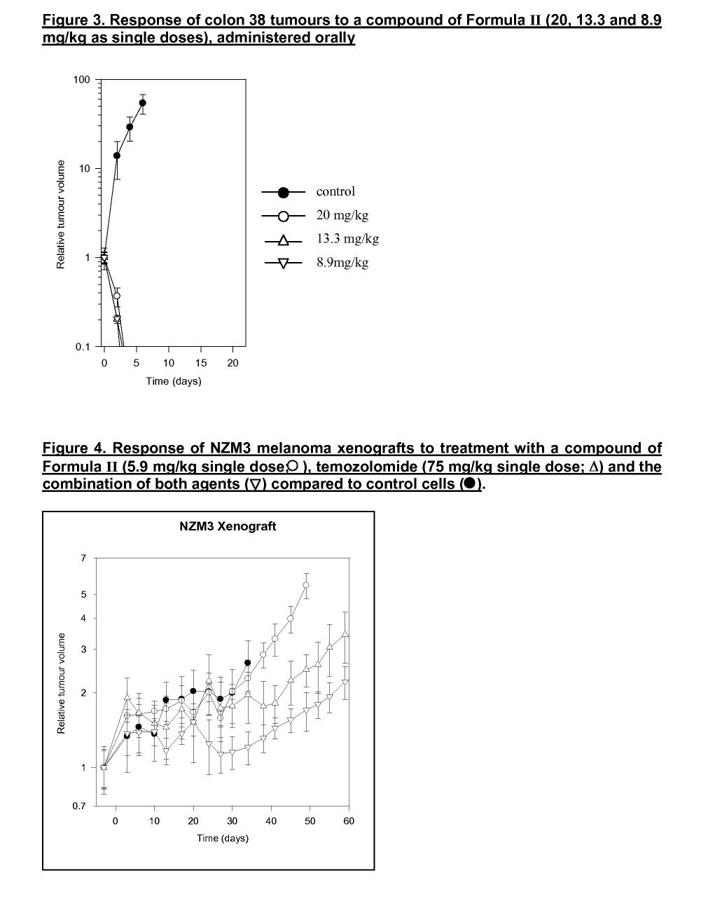

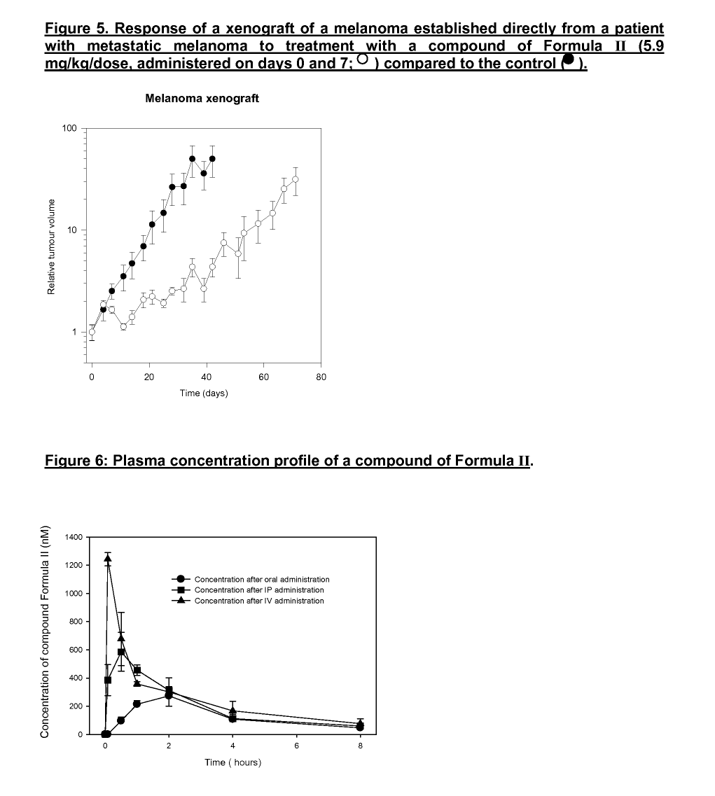

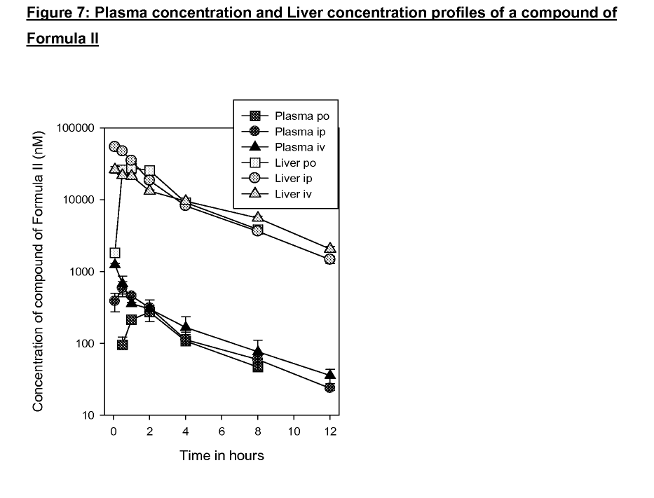

Melanomas respond poorly to cytotoxic agents but a proportion respond to the DNA methylating agent temozolomide. In order to test the ability of the compound of Formula II to combine with other drugs, xenografts of the NZM3 melanoma line were grown in immunodeficient (nude) mice and treated simultaneously with single doses of the compound of Formula II together with the anticancer drug temozolomide. The results indicate that combination treatment of a melanoma xenograft is both feasible and effective (FIG. 4).

### Example 10

### Response of a Melanoma Taken Directly from a Cancer Patient

Metastatic melanoma is particularly resistant to current chemotherapy. Melanoma tissue taken from a patient at surgery was implanted subcutaneously in immunodeficient mice. After three months, the tumour in one animal was excised and transferred to further immunodeficient mice. The tumours were grown to a diameter of approximately 4 mm before treatment and the compound of Formula II was administered in two doses of 5.9 mg/kg, 7 days apart. The results show strong inhibition of melanoma growth (FIG. 5).

### Example 11

### Plasma Pharmacokinetics

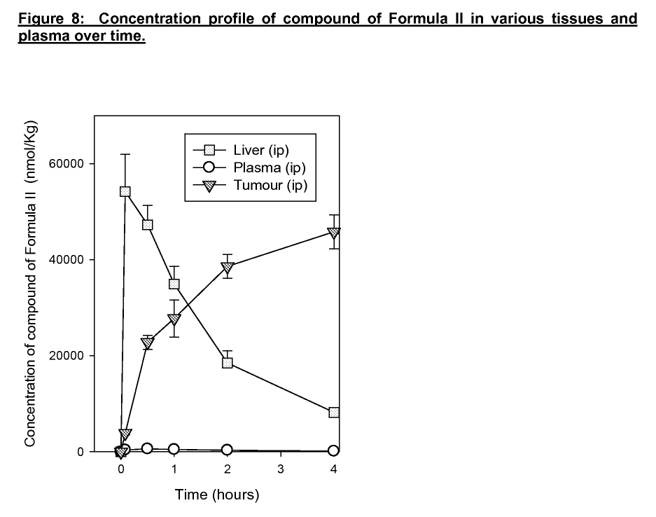

The plasma pharmacokinetics of the compound of Formula II, were compared following intravenous, intraperitoneal and oral administration at a dose of 8.9 mg/kg (26 μmol/kg). Mice were terminally anaesthetised at various times up to 8 hours after drug administration and blood was taken for preparation of plasma samples. Plasma concentrations of the compound of Formula II were measured by high performance liquid chromatography-mass spectrophometry. The results are shown in FIG. 6, the vertical bars showing the standard deviation of the mean values from three mice per time point. The maximum plasma concentrations achieved were 0.27 μM for oral administration (achieved at 2 hours) 0.57 μM for intraperitoneal administration (achieved at 0.5 hours) and 1.24 μM for intravenous administration (achieved at 0.08 hours). The areas under the plasma concentration-time profile for the first 8 hours (AUC values) were 1030 μM.h for oral administration, 1631 μM.h for intraperitoneal administration and 1994 μM.h for intravenous administration. The terminal plasma half life for a compound of Formula II, for all three routes of administration was approximately 3.5 hours. The results show that the compound of Formula II shows good dose potency and reproducible bioavailability following oral administration (63% of that of intraperitoneal administration).

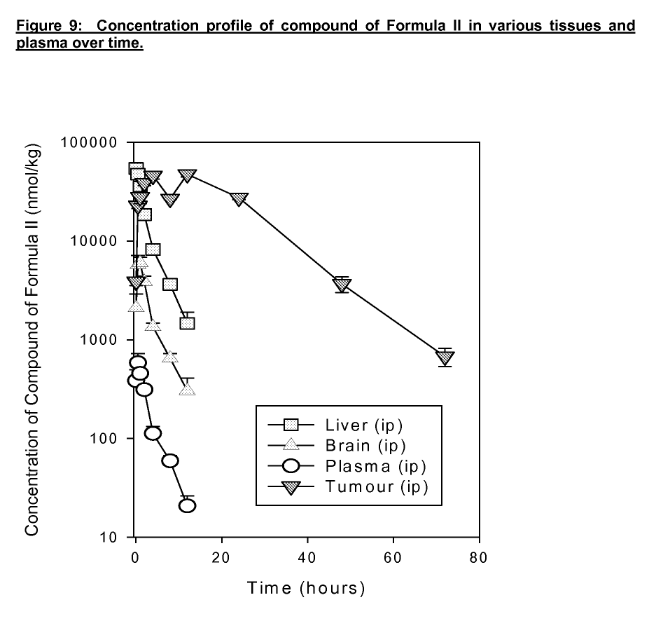

In a further comparison, following intraperitoneal (i.p.), intravenous (i.v.) and oral (p.o.) administration in non tumour bearing mice and using the same methods as above, the time-concentration profiles for the different routes of administration are quite comparable (FIG. 7). As expected from the rate of gastric emptying, the initial increase in plasma and liver drug concentrations occur later after oral administration than they do after intravenous or intraperitoneal administration, but the plasma and tissue half-lives are similar.

### Example 12

### Plasma and Tissue Pharmacokinetics

Plasma and tissue pharmacokinetics of the compound of Formula II, were compared following intraperitoneal administration at a dose of 8.9 mg/kg body weight. Mice were terminally anaesthetised at various times up to 75 hours after drug administration and tissue and blood was taken for preparation of samples. Plasma and tissue concentrations of the compound of Formula II were measured by high performance liquid chromatography-mass spectrophometry. The results are shown in FIG. 8, the vertical bars showing the standard deviation of the mean values from three mice per time point. The concentration of drug in plasma is very low on this scale (<100 nmol/kg), implying a high ratio of the concentration of cell-associated drug to that of external drug

Plasma and tissue pharmacokinetics, were further investigated following intraperitoneal administration of the compound of Formula II at a dose of 8.9 mg/kg. Mice were terminally anaesthetised at various times up to 75 hours after drug administration and tissue and blood was taken for preparation of samples. Plasma and tissue concentrations of the compound of Formula II were measured by high performance liquid chromatography-mass spectrophometry. The results are shown in FIG. 9, the vertical bars showing the standard deviation of the mean values from three mice per time point. The concentration differential (ie the slope of each plot) indicates the half life of the compound of Formula II in each tissue type and plasma. The half life of the compound of Formula II in various tissue types including liver, kidney, brain, heart and plasma, calculated from points on the concentration time curve, is approximately three hours. The half life of the compound of Formula II in tumour tissue, calculated from points on the concentration time curve, is approximately 9 hours. This is approximately a three-fold increase in the concentration differential in tumour tissue to non-tumour tissue and plasma concentration of Formula II. This effectively leads to an accumulation of the compound of Formula II in tumour tissue relative to other tissue.

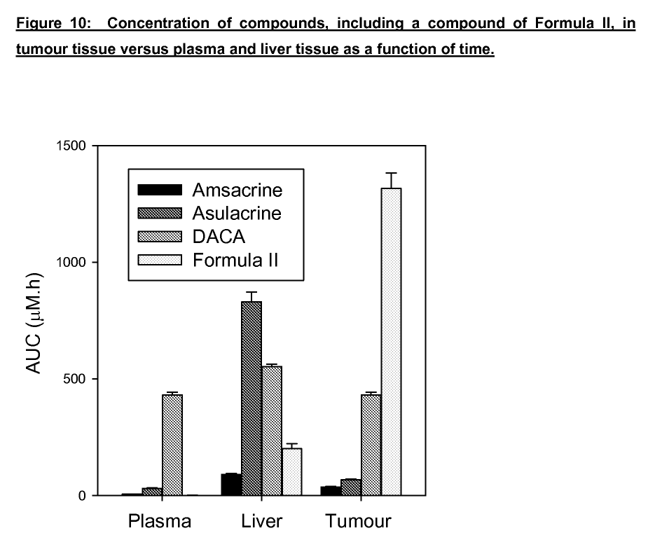

The plasma and tissue pharmacokinetics of a compound of Formula II, were also compared with several well known cancer therapies as shown in FIG. 10. The analytical methods used to obtain data for other therapies varies and was taken from the literature. The AUC (area under the concentration-time curve) values of the compound of Formula II show approximately a 6-7 fold increase in concentration of Formula II in tumour tissue over liver tissue. The AUC values in plasma are negligible. A longer half life (higher concentration differential) in a particular tissue, leads to a larger AUC value (area under the concentration versus time curve). The AUC values provide an indication of the amount of each compound accumulated in a particular tissue or plasma over time. In contrast, amsacrine and asulacrine appear to accumulate more in the liver tissue than tumour tissue and DACA is relatively evenly spread throughout each tissue and plasma.

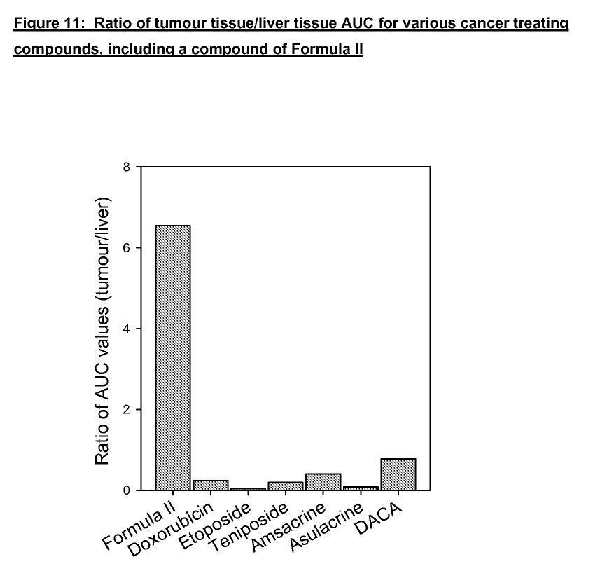

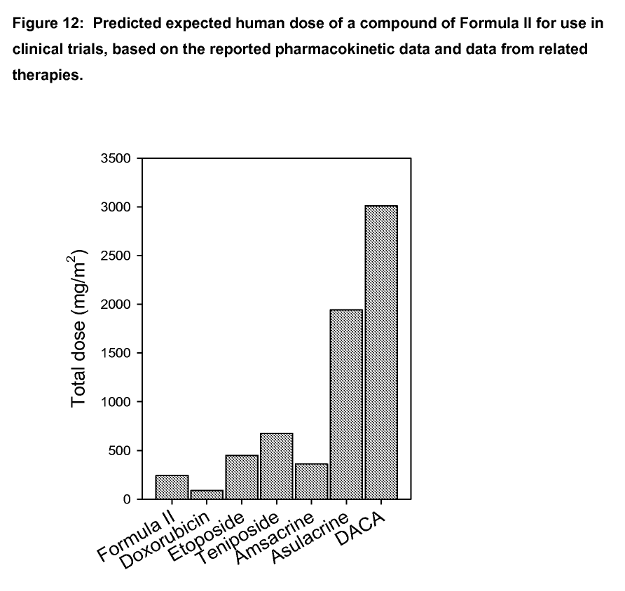

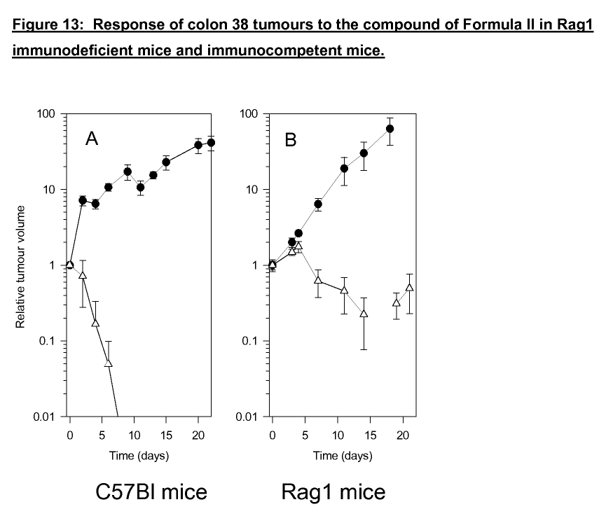

The ability of different anticancer drugs to be retained selectively in tumour tissue can also be conveniently compared as the ratio of AUC values for a normal tissue versus a tumour tissue. Most published studies use liver as the normal tissue, and values of this ratio (tumour/liver) are shown in FIG. 11. The ratio for the compound of Formula II is clearly superior to that of the compared drugs, including doxorubicin Shinkai H, Takahashi H, Miyamoto K, Uchida T, Tokiwa T, Comparative pharmacokinetics of KRN8602, a new morpholino anthracycline, and adriamycin in tumor-bearing mice. Cancer Chemother Pharmacol. 1996; 38: 417-24), amsacrine and asulacrine (Kestell P, Paxton J W, Evans P C, Young D, Jurlina J L, Robertson I G C, Baguley B C. Disposition of amsacrine and its analogue 9-((2-methoxy-4-[(methylsulfonyl)-amino]phenyl)amino)-N,5-dimethyl-4-acridinecarboxamide (CI-921) in plasma, liver, and Lewis lung tumors in mice. Cancer Res 1990; 50: 503-508) and DACA (Kestell P, Dunlop I, McCrystal M R, Evans B D, Paxton J W, Gamage R S K A, Baguley B C. Plasma pharmacokinetics of DACA (N-[2-(dimethylamino)-ethyl]-acridine-4-carboxamide) in a phase I trial. Cancer Chemother Pharmacol 1999; 44: 45-50). Similar analytical methods, employing high performance liquid chromatography, were used.
The known DNA binding properties of compound of Formula II, together with the pharmacokinetic observations suggesting that most of the administered drug is present on the DNA or normal and tumour tissue, can be used to predict the expected human dose in clinical trials. This is shown in FIG. 12. Although this dose of up to approximately 300 mg/m² (which might typically be administered once every 3-4 weeks) is somewhat higher than that used for doxorubicin, it compares favourably with clinical doses used for other standard drugs as well as for the investigational drugs asulacrine and DACA. Note: the dosage units relate to weight of drug per square meter of skin of the subject. A typical subject may have 1.7 m² of skin.

### Example 13

### Response of Immunodeficient Mouse Model

A single dose of 5.9 mg/kg of the compound of Formula II was administered intraperitoneally to immunocompetent mice in A, while a 5.9 mg/kg dose on days 0 and 7 was administered to Rag 1 immunodeficient mice (see FIG. 13). The compound of Formula II was found to be active against colon 38 tumours in immunocompetent mice, while the affect is attenuated in Rag1 immunodeficient mice. Initial tumour response (apoptosis induction) is similar in normal and immunosuppressed mice. The compound of Formula II appears to induce a change in tumour cells that activates a T-cell mediated response leading to tumour regression. This dependence on T-cells for response might explain why only moderate activity of the compound of Formula II is found in xenografts.

### Discussion

The compounds of Formula I according to the present invention are chemically distinct from either DACA, which has an acridine chromophore, or the compound XR 11576, which has a phenazine chromophore.

The compounds of the present invention are water soluble, easy to formulate and stable.

In vitro, the compounds of the present invention are active against a multidrug resistant cell line, suggesting that the compounds of the present invention may overcome multidrug resistance.

In vitro, the compounds of the present invention are highly effective inducers of the p53 pathway, which is known to lead to susceptibility to apoptosis induction.

In vitro, the compounds of the present invention are highly effective inducer inhibitors of survivin expression. Survivin is known to act as an anti-apoptotic signal, enhancing cell survival.

The compounds of the present invention are highly dose-potent in vivo with a maximum tolerated single dose of 8.9 mg/kg in conventional mice and 5.9 mg/kg in nude mice. The preferred compound of Formula II, has oral activity against the murine colon 38 carcinoma, inducing single dose cures. This is an unexpected feature of a compound of Formula II. The pharmacokinetics of the compound following intraperitoneal (i.p.), intravenous (i.v.) and oral (p.o.) administration demonstrate the time-concentration profiles for the different routes of administration are quite comparable.

The compounds of the present invention have the property of being taken up relatively efficiently by tumour tissue but, more importantly, to be retained selectively by tumour tissue in comparison with normal tissues. The tissue half-life in mice is approximately 3 hours in plasma, liver, kidney, brain and heart tissue, but is approximately 9 hours in colon 38 tumour tissue.

The known DNA binding properties of compounds of the present invention, together with the pharmacokinetic observations suggest that most of the administered drug is present on the DNA of normal and tumour tissue and can be used to predict the expected human dose in clinical trials. Although this dose of up to 450 mg/m² (which might typically be administered once every 3-4 weeks) is somewhat higher than that used for doxorubicin, it compares favourably with clinical doses used for other standard drugs as well as for the investigational drugs asulacrine and DACA.

The compounds of the present invention have a spectrum of activity against human tumour lines grown as xenografts. As measured by the NCl criteria of T/C<40%, they were active in 6 of 9 xenografts and showed excellent activity against a melanoma transplanted directly from a patient to an immune-deficient mouse (T/C<10%; growth delay >30 days).

Where in the foregoing description reference has been made to integers having known equivalents thereof, then those equivalents are herein incorporated as if individually set forth.

While this invention has been described with reference to certain embodiments and examples, it is to be appreciated that further modifications and variations may be made to the embodiments and examples without departing from the spirit or scope of the invention.

What we claim is:

1. A composition that provides up to 450 mg/m² of a compound of Formula I: 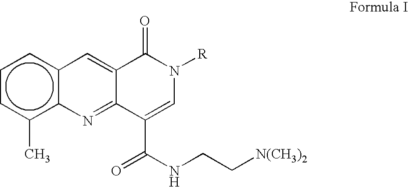
wherein R is selected from a C₁-C₆ alkyl, unsubstituted phenyl or phenyl substituted by halo, C₁-C₆ alkyl or C₁-C₆ alkoxy or an enantiomer, racemate, isomer or physiologically acceptable salt thereof.

2. A composition according to claim 1, wherein R is selected from methyl, ethyl, butyl, unsubstituted phenyl, 4-fluoro-phenyl or 3,4-dimethoxy-phenyl.

3. A composition according to claim 1, wherein R is methyl.

4. A composition according to claim 1, wherein the composition is formulated for oral, intraperitoneal or intravenous administration.

5. A composition according to claim 1, wherein the composition provides up to 300 mg/m² of a compound of formula I.

6. A composition according to claim 1, wherein the composition includes a pharmaceutically acceptable excipient, adjuvant, carrier, buffer or stabiliser.

7. A composition including a compound of Formula I: 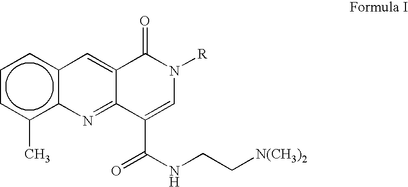
wherein R is selected from a C₁-C₆ alkyl, unsubstituted phenyl or phenyl substituted by halo, C₁-C₆ alkyl or C₁-C₆ alkoxy or an enantiomer, racemate, isomer or physiologically acceptable salt thereof, wherein the composition provides at least a 3-fold concentration differential in tumour tissue to non-tumour tissue or plasma concentration of compound of Formula I.

8. A composition according to claim 7, wherein the composition is adapted to provide about a 7-fold concentration differential.

9. A composition according to claim 7, wherein the composition is suitable for use in a dosage regimen to maintain at least a 3-fold concentration differential in tumour tissue to non tumour tissue or plasma concentration of compound of Formula I.

10. A composition according to claim 1, further including one or more chemotherapeutic agents.

11. A composition according to claim 7, further including one or more chemotherapeutic agents.

12. A composition according to claim 1, further including one or more chemotherapeutic agents selected from:
temozolomide or other DNA methylating agents;
cisplastin or other platinum-based derivatives;
cyclophosphamide or other DNA-alkylating agents;
doxorubicin, mitoxantrone, camptothecin or other topoisomerase inhibitors;
methotrexate, gemcitabine or other antimetabolites;
docetaxel or other taxanes; and
kinase inhibitors

13. A composition according to claim 1 wherein in the compound of Formula I, R is methyl and the composition further includes the chemotherapeutic agent temozolomide.

14. An oral composition including a compound of Formula I as defined in claim 1, together with a suitable oral carrier.

15. An oral composition according to claim 14, further including one or more chemotherapeutic agents.

16. A method of treating and/or preventing abnormal or aberrant cell growth in a subject including the step of administering to a subject in need thereof a composition that provides up to 450 mg/m² of a compound of Formula I: 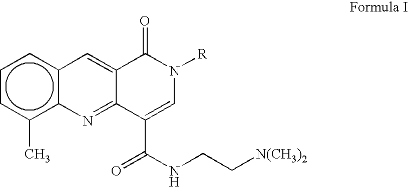
wherein R is selected from a C₁-C₆ alkyl, unsubstituted phenyl or phenyl substituted by halo, C₁-C₆ alkyl or C₁-C₆ alkoxy or an enantiomer, racemate, isomer or physiologically acceptable salt thereof.

17. A method according to claim 16, wherein the abnormal or aberrant cell growth is a cellular proliferative disorder.

18. A method according to claim 16, wherein the proliferative disorder is multidrug resistant.

19. A method according to claim 16, wherein the composition is formulated for oral administration.

20. A method of treating a subject with a multidrug resistant cellular proliferative disorder, including the step of administering to a subject in need thereof a composition as defined in claim 1 which further includes one or more chemotherapeutic agents.

21. A method of treating and/or preventing abnormal or aberrant cell growth in a subject including the step of administering to a subject in need thereof a composition including a compound of Formula I as defined in claim 1; wherein the composition provides at least a 3-fold concentration differential in tumour tissue to non tumour tissue or plasma concentration of compound of Formula I.

22. A method according to claim 21, wherein the composition is adapted to provide about a 7-fold concentration differential.

23. A method of inducing p53 expression and decreasing survivin expression in cells demonstrating abnormal or aberrant growth, by exposing the cells to a compound of Formula I, whereby the exposure comprises administering to a subject in need thereof a composition including a compound of Formula I as defined in claim 1 in an amount sufficient to induce p53 expression and decrease survivin expression in the subject having a cellular proliferative disorder.

24. A method of claim 23 further comprising exposure of the cells to a combination of the compound of Formula I and one or more chemotherapeutic agents.

25. A method of claim 23 comprising the combination of a compound of Formula II and temozolomide.
